(19) United States
(12) Patent Application Publication
(10) Pub. No.: US 2007/0213638 A1
(43) Pub. Date: Sep. 13, 2007

(54) Device and Method for Drawing a Sample and Analyzing Body

(76) Inventors: Peter Herbrechtsmeier, Konigstein (DE); Wolfgang Ehrfeld, Mainz (DE); Karoly Nagy, Aachen (DE); Stefan Kammermeier, Nurnberg (DE); Alexander Azzawi, Mainz (DE); Egbert Linnebach, Buttelborn (DE); Thomas Schwank, Columbus, IN (US)

Correspondence Address: K.F. ROSS P.C., 5683 RIVERDALE AVENUE, SUITE 203 BOX 900, BRONX, NY, 10471-0900, US

(21) Appl. No.: 11/578,758

(22) PCT Filed: Jun. 25, 2004
(86) PCT No.: PCT/EP04/06908

§ 371 Date: Oct. 16, 2006

(30) Foreign Application Priority Data
Jun. 27, 2003 (EP) 03014772.2
Feb. 4, 2004 (EP) 04002436.6

(51) Int. Cl. A61B 5/157

(52) U.S. Cl. 600/583

(57) ABSTRACT

The invention relates to a device for drawing a sample and analyzing body fluids, and to a corresponding method. The device comprises a handle (1), which accommodates a pneumatic drive device, and a sensor unit (2) that contains a microneedle (3) on an elastic membrane (4) and at least one sensor system (5). The handle (1) and the sensor unit are detachably connected to one another, and the pneumatic drive device interacts with the sensor unit (2) in such a manner that the drive device exerts pressure onto the elastic membrane (4) so that the microneedle (3) pierces the surface of the skin in order to draw body fluid. During the ensuing decreasing pressure, the elastic membrane (4) returns to its original shape, and body fluid is suctioned, due to the resulting underpressure, from the location of piercing and into the interior of the sensor unit (2) to the at least one sensor system (5). Particularly advantageous is the integration of a measuring unit (10), which determines concentration values resulting from the physical or chemical property changes arising on the sensor system (5). A display (12) can serve to display the measured values.

The object of the invention is an apparatus and a corresponding method that can be used to draw a sample of bodily fluid, for example blood, and carry out the quantitative analysis of the components thereof.

In clinical diagnostics, the analysis of bodily fluids, particularly of blood, is an important method used to study the health of a patient. The most frequent analyses are carried out in the home-care sector by the patient himself using capillary blood. For these applications, particularly for the determination of blood glucose levels, patients use lancing aids in order to slightly injure the skin and obtain a small drop of blood. This blood sample is typically applied to a test strip that is evaluated in a measuring apparatus. So as to simplify this complex procedure and minimize the patient's pain, numerous methods and technologies have been developed. It has been attempted to carry out multiple steps using a single apparatus and furthermore to reduce the volume of blood required for analysis. The latter goal can be achieved, for example, in that the diameter of the lancing needle is reduced and the lancing depth is precisely adjustable. A thin lancing needle on the other hand produces only a small injury of the skin, so that only a little or no blood at all is obtained. Additional auxiliary measures, such as pressing the skin together or periodically stimulating the skin at the piercing site, meaning with vibration, as well as the suction effect of a vacuum that is produced to allow the amount of blood that is obtained to be increased.

U.S. Pat. No. 5,505,212 describes an apparatus that is used to draw the blood sample into a chamber. The chamber has a flexible and spherical top wall in which a needle is positioned. During use, the needle is pushed through the wall downward into the skin. At the same time, the spherical wall is likewise pushed down. When the pressure is relieved from above, the apparatus returns to the starting position, thus producing a vacuum in the chamber that assists the process of drawing blood into the chamber. The suction effect created by a vacuum for drawing in blood has been known for years, see for example U.S. Pat. No. 4,653,513. The disadvantage of the apparatus described in U.S. Pat. No. 5,505,212 is that the chamber and the spherical top wall have to have relatively large dimensions in order to be able to produce any noticeable suction effect. Consequently, a relatively large amount of blood is drawn, at least several microliters, since by the end of the sampling process the chamber is completely filled with blood, and a smaller amount cannot be taken. The patent includes no information about a drive mechanism and a measuring apparatus that may potentially be used.

WO 02/100253 describes an apparatus and a method that are used to draw blood samples in a sealed configuration. The drawing of the blood sample is helped by a vacuum. From a functional point of view, the apparatus is designed for relatively large sample volumes, meaning 1 to 5 microliters. Another possibility that is mentioned in this patent is the integration of a sensor layer.

DE 37 08 031 A1 describes a sensor layer that is integrated in a cannula. The cannula with the sensor layer is part of a suction cup, and reduced pressure is created by heating and subsequently cooling a reservoir. Optical measuring is carried out in a complex fashion using optical fibers, the measuring apparatus not being integrated in the apparatus.

US 2003/0055326 describes a microneedle for drawing bodily fluids and measuring test samples. An electrochemical sensor unit is inserted concentrically in the microneedle, meaning a micro-cannula measuring less than 350 micrometers in diameter. The drawing of the fluid is supported by capillary forces. A concept of this type is prone to error. For example, beneath the sensor unit in the microneedle an enclosed space exists that cannot be filled by capillary forces, or can be filled only partially, since an air bubble always remains trapped in it.

A compact apparatus is disclosed in U.S. Pat. No. 4,637,403. The sensor unit is provided on the end of a needle or a capillary. Visual methods are used for evaluation. For collecting the blood, a vacuum is produced. A microprocessor assumes the control and logistics of the measuring procedure and displays the reading. The disadvantage of this concept is the relatively long path of the blood to the sensor.

A compact measuring apparatus is also revealed in US 2002/0198444. An electrical or optical sensor unit as well as a microprocessor for control and logistics purposes are integrated in the apparatus. The needle and the movable test strip are two separate units that are installed in the lance mount. Here, the blood is not drawn into the apparatus, but instead the test strip is moved to the drop of blood. Due to the complex design of the lancet mount and the guidance of the movable parts, this single-use part is expensive to produce. Since the lancet mount is not intended to serve as a reservoir for the blood, functional errors can also easily occur, for example when the apparatus slips on the skin.

For some time now, lancets supported by a vacuum are known in a variety of versions, by way of example in U.S. Pat. No. 4,653,513, EP 0 622 046, EP 0 838 195, U.S. Pat. No. 6,332,871, U.S. Pat. No. 6,086,545 and EP 1 060 707. The general characteristic of these finger prickers is that they work with standard lancets or very similar lancets thereto, and there is no integrated a sensor or test strip. U.S. Pat. No. 6,332,871 only mentions the possibility that a test strip may be inserted in the apparatus laterally of the needle. Also, the space for drawing in blood is relatively large in these lancets, and a comparatively long plunger displacement is required for efficiently creating a vacuum.

In WO 03/094752 and WO 02/100254, the piercing parameters of the needles are adjusted electronically, and the lancet is selected by electromechanical components. The reference mentions the possibility of integrating sensors and detectors, however these solutions are not explained in detail. The use of electronic aids could increase comfort during collection of the samples. However, this is associated with high manufacturing costs, complex power supply and therefore a relatively large and bulky apparatus.

Recently, numerous inventions are known in which the needle and sensor are integrated in one unit. Such a unit with a test element that is not specified in detail is known from EP 1 287 785, a unit with an electric sensor is revealed in U.S. Pat. No. 6,607,658, and a unit using an optical-measurement method is known from EP 1 342 448.

Compact apparatuses with integrated measuring units are disclosed in U.S. Pat. No. 6,352,514, EP 1 362 551, EP 1 360 934, EP 1 360 933, WO 03/088834 and WO 02/101359. These apparatuses utilize capillary force for pumping the blood to the sensor. With the exception of U.S. Pat. No. 6,352,514, drive and control are electronic. While handling is simplified by the electronics, the electromechanical actuators are more prone to malfunction than purely mechanically operating drive systems.

U.S. Pat. No. 6,506,168 and U.S. Pat. No. 6,306,104 describe compact measuring apparatuses with integrated lancets, sensor units and measuring systems. The apparatuses are fully electronic and use a vacuum pump for pumping the blood. The disadvantage in both cases is that the test strip and the lancet are provided separately in the apparatus and have to be replaced individually. This cumbersome procedure negatively affects the comfort aspect and increases the sources of error and risk of contamination. Furthermore, the use of pumps for producing a vacuum increases manufacturing costs and requires a complex power supply.

The disadvantages of the lancets, sensory systems and integrated apparatuses mentioned above can be summarized as follows: relatively large amounts of bodily fluid, typically blood, are required, no pain-free lancing, high manufacturing costs, lacking integration of the individual components and steps, lacking hygiene and comfort, large and heavy designs due to high power supply.

The object of the present invention is to improve an apparatus and a corresponding method that can be used for drawing a sample of a bodily fluid, for example blood, and quantitatively analyzing the components therein, such that maximum comfort is achieved for the user at a low cost.

The object is achieved according to the invention by an apparatus according to claim 1 and a corresponding method.

The apparatus according to the invention for drawing a sample and analyzing bodily fluids, particularly blood, comprises a handle that accommodates a pneumatic actuator, and a sensor unit with a microneedle that is firmly connected to an elastic membrane, and with at least one sensor system. The handle and the sensor unit are detachably connected to one another, and the actuator interacts with the sensor unit such that the pneumatic actuator exerts a certain amount of pressure on the elastic membrane via a compressed air chamber so that the microneedle pierces the surface of the skin in order to draw bodily fluid. Thereafter, the pressure on the elastic membrane is decreased. As the membrane returns to its original shape, reduced pressure is produced in the interior of the sensor unit and the bodily fluid is transported from the piercing location to at least one sensor system. In the sensor system, the components of the bodily fluid to be determined cause a physical or chemical property change of the sensor system.

The apparatus according to the invention has the advantage that it is comparatively inexpensive to produce. The pneumatic actuator for piercing of the skin with the microneedle and the subsequent drawing of bodily fluid is based on a simple mechanical principle, is easy to handle and not very prone to malfunction. By appropriately dimensioning the interior of the sensor unit, the required sample volume of bodily fluid can be optimally reduced. The use of a microneedle, assisted by a suction effect for improving blood collection, keeps pain to a minimum. A particularly advantageous feature is the detachable connection of the handle and sensor unit, since this way a corresponding sensor unit can be configured as a sterile disposable item that can be produced cost effectively, for example as an injection-molded part.

Advantageous embodiments are disclosed in the dependent claims.

In a preferred embodiment of the apparatus, the handle comprises a measuring unit for determining the physical or chemical property changes caused in the sensor system by the effects of the components of the bodily fluid that are to be determined. The measuring unit uses these property changes to determine the concentration value of a component to be determined. Advantageously, the information is displayed on a display integrated in the apparatus. This way it is possible to draw a sample, determine the desired analytical value and display the value directly to the user in a single process.

It is also possible to store readings. In a special embodiment, the results can also be forwarded to a second apparatus using a transponder or similar auxiliary unit. This second apparatus can be, for example, an automatic syringe or a pump for administering appropriate therapeutic agents, such as insulin. The apparatus can be a display apparatus, such as a mobile telephone, a watch, a computer or a PDA.

Since the analysis of bodily fluids should be carried out cost efficiently, reliably and quickly, optical measuring methods for the sensor system, particularly fluorescence spectroscopic measuring methods, are particularly advantageous. For fluorescent measurement, a minimal surface on the sensor system may be used, thus making the production of the sensor units cost effective. In a preferred embodiment, the detection method used is the FRET (fluorescence resonance energy transfer) method.

The quantitative evaluation of the biochemical reaction occurring in the sensor system may be carried out with optical methods, such as UV/VIS absorption measurement, SPR (surface plasmon resonance), IR spectroscopy, ellipsometry, colorimetry, fluorescence spectrometry or also electrochemical or other determination methods. Possible electrochemical measurement methods include amperometry and coulometry. Other methods use SAW (surface acoustic waves), oscillating crystals, impedance measurements and cantilevers.

A further advantageous embodiment of the apparatus according to the invention allows the simultaneous quantitative determination of several components of the bodily fluid using the same or different measurement methods. For this, several sensor systems are accommodated in the sensor unit. While this requires a slightly greater volume of blood, the spatially close arrangement of the sampling site to the sensor system means that the amount of blood used is significantly less than with conventional methods, in which considerably more blood is collected and has to be transported to remote measuring apparatuses. In the case of fluorescence measurement, particularly when using the FRET method, it is even possible to measure several test samples on the same surface of a sensor system, because the different detection systems can be mixed on one sensor system due to measurements at different excitation wavelengths.

The detachable connection between the handle and the sensor unit is advantageously configured as a quick-release connection. It can be, for example, a plug, clamp, snap-fit, screw or adhesive connection. In any case, the important aspect is that the connection can be detached or established quickly and easily and that the connection of the handle to the sensor unit produces a stable apparatus.

In a further embodiment, the sensor unit comprises a control unit for detecting the correct, particularly sufficient, filling level of bodily fluid. So as to reliably analyze the sample, it is required that in any case a sufficient amount of bodily fluid be available. The corresponding control unit may include an electrical resistivity measuring unit with piezoelectric or electromagnetic elements for power supply purposes. Sufficient filling for a reliable analysis may be, for example, 0.5 ml or less. This amount can be specified as the target value in the control unit.

It is provided in another embodiment that the handle comprises a first adjustment member for variably defining the piercing depth of the microneedle. This is particularly advantageous when the piercing depth should be adjusted according to the piercing location on the skin. Therefore, it will depend on the location of the piercing whether the skin surface is thicker or thinner and whether the blood vessels are located closer to the surface or deeper down.

Furthermore, it is possible to provide a second adjustment member on the handle for variably defining the piercing speed. This way, individual adjustment is possible, allowing minimization of the pain. A corresponding possibility exists for the variable definition of the collection speed of the bodily fluid by the microneedle by using a third adjustment member.

For analyzing the bodily fluid with the sensor system, it may be advantageous to provide an upstream filter arrangement. It may potentially also be necessary to subject the bodily fluid first to special processing. This step may be carried out by using reagents and/or rinsing solutions from reservoirs that may also be provided upstream of the sensor system and process a bodily fluid appropriately for sampling.

The method for drawing a sample and analyzing bodily fluids comprises several steps. In a first step, a pneumatic actuator accommodated in a handle exerts pressure via a compressed air chamber on an elastic membrane in a sensor unit so that the microneedle attached to the elastic membrane pierces the surface of the skin and the microneedle penetrates the skin surface. Thereafter, the pressure on the elastic membrane is decreased. As the membrane returns to its original shape, reduced pressure is produced in the interior of the sensor unit and the bodily fluid is transported from the piercing location to at least one sensor system. In the sensor system, the components of the bodily fluid to be determined cause a physical or chemical property change of the sensor system.

In a next step, the measuring unit uses these property changes to determine a concentration value of the component to be determined and optionally shows it on a display.

Advantageously, an apparatus according to the invention is used for a method of this type.

The invention will be explained in more detail hereinafter with reference to the figures below. Therein:

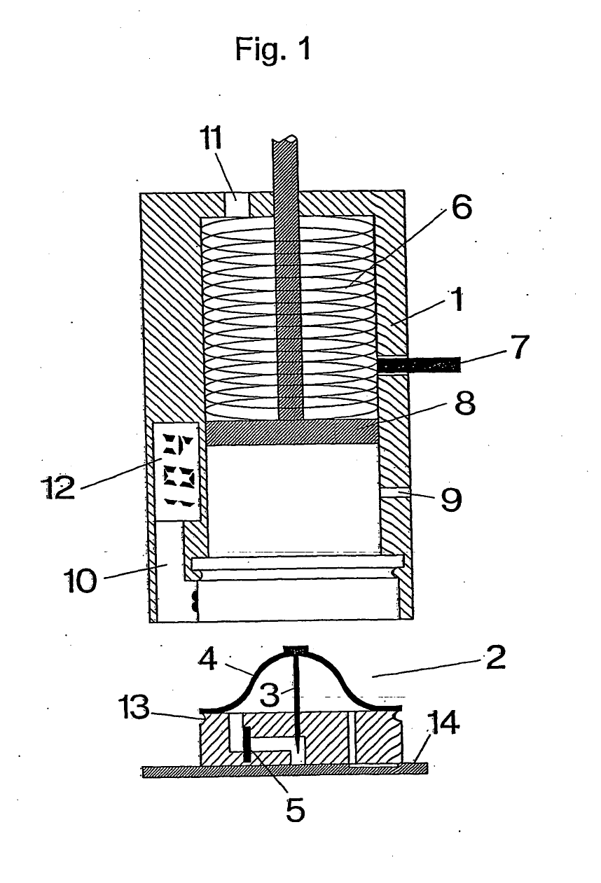

FIG. 1 is a longitudinal sectional schematic illustration of an apparatus according to the invention;

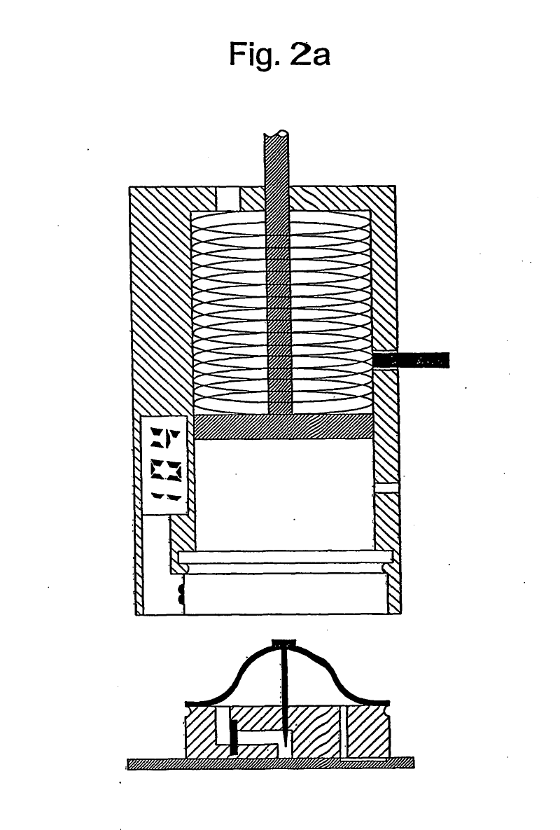

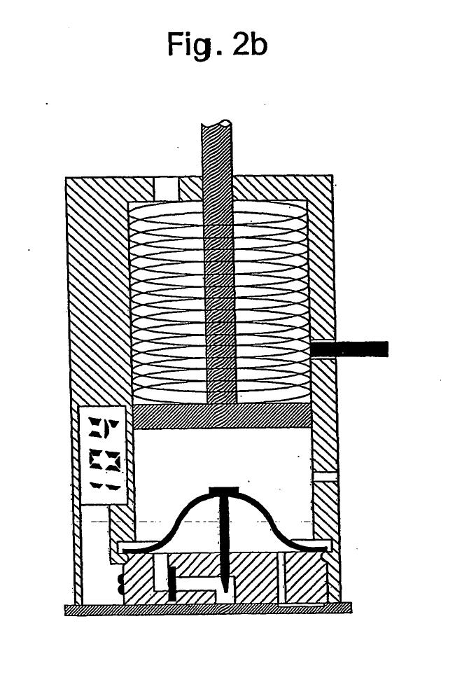

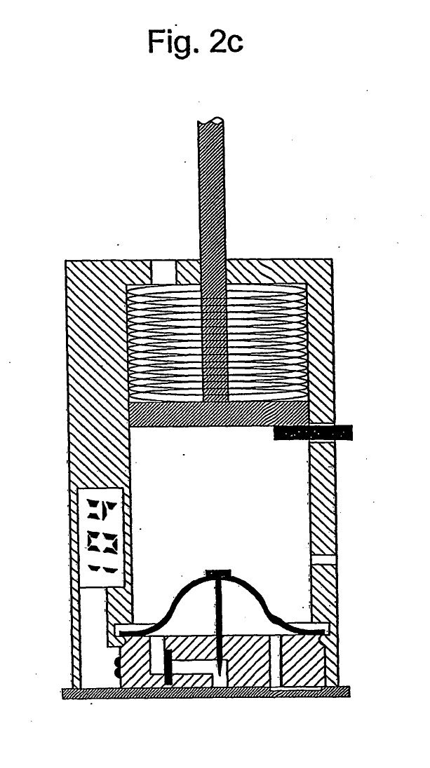

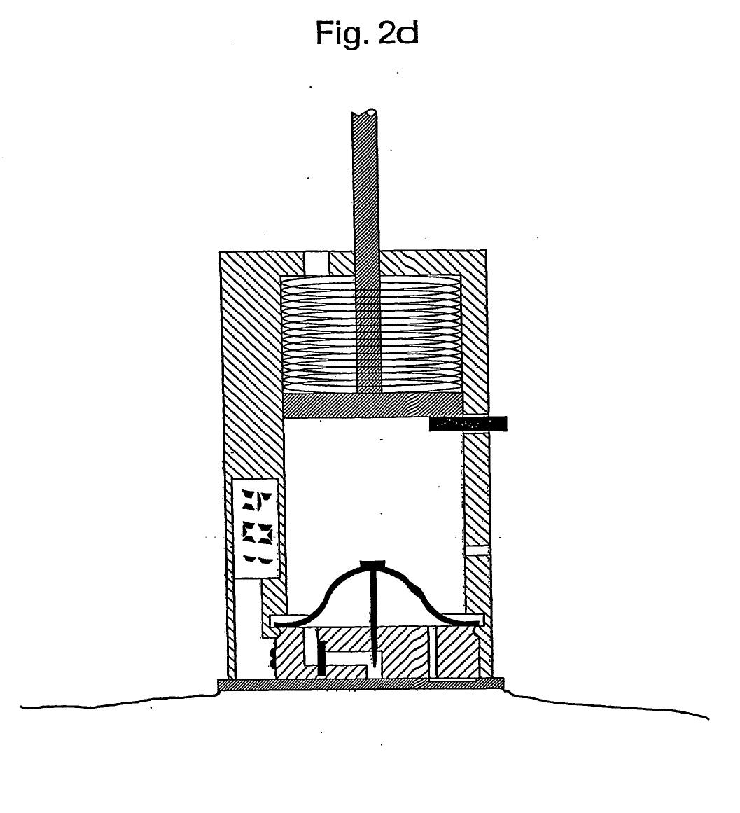

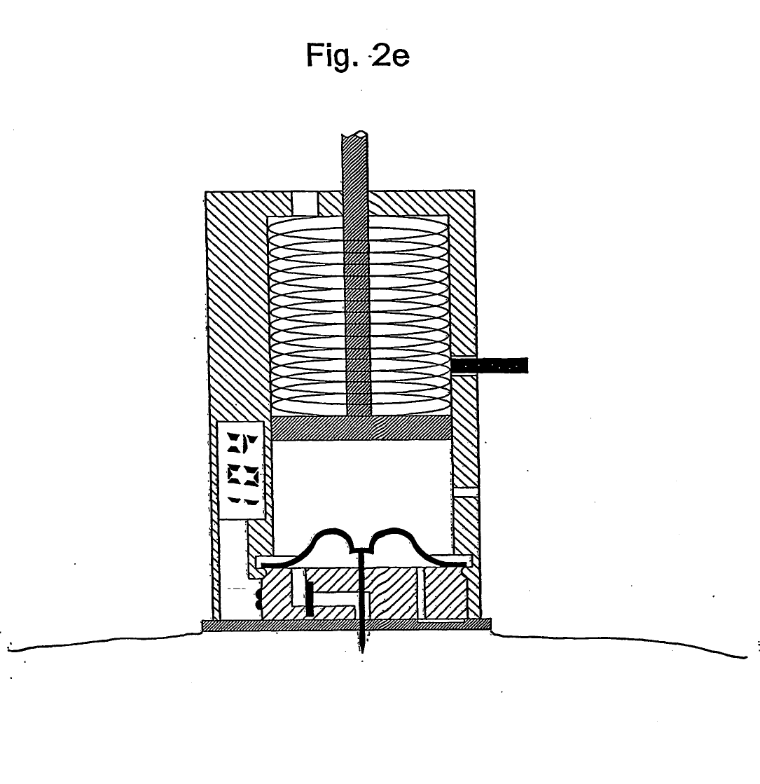

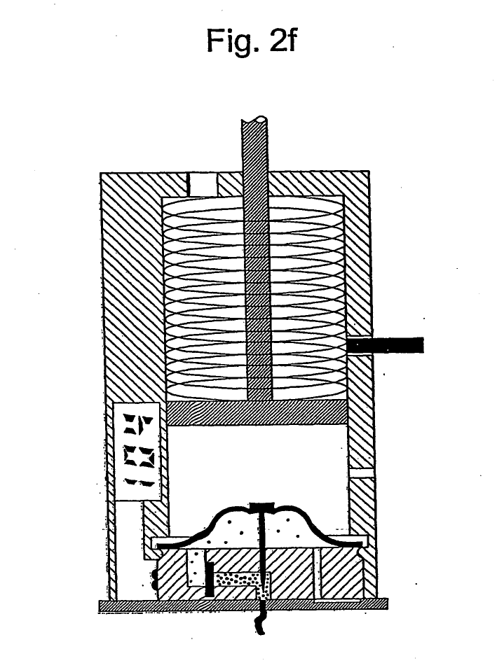

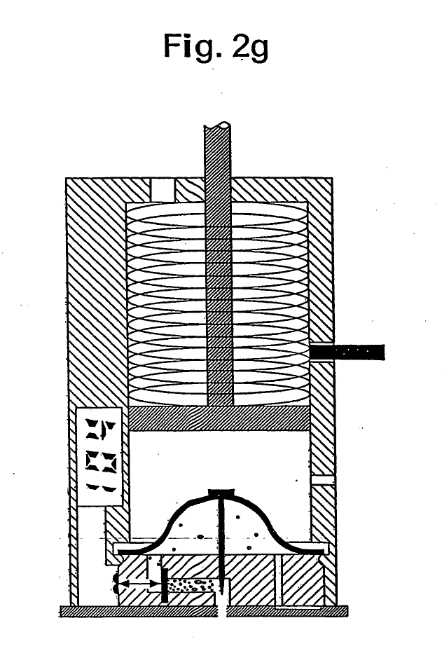

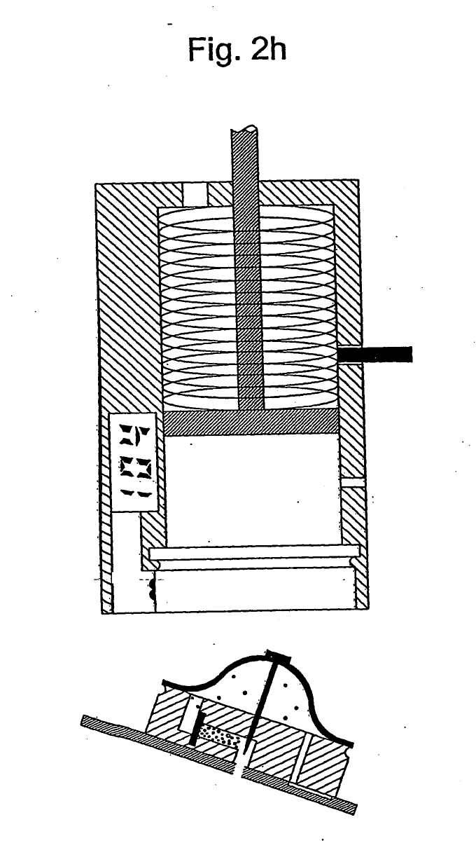

FIGS. 2a to 2h illustrate drawing a sample and analyzing bodily fluids using the apparatus according to FIG. 1 in eight consecutive phases;

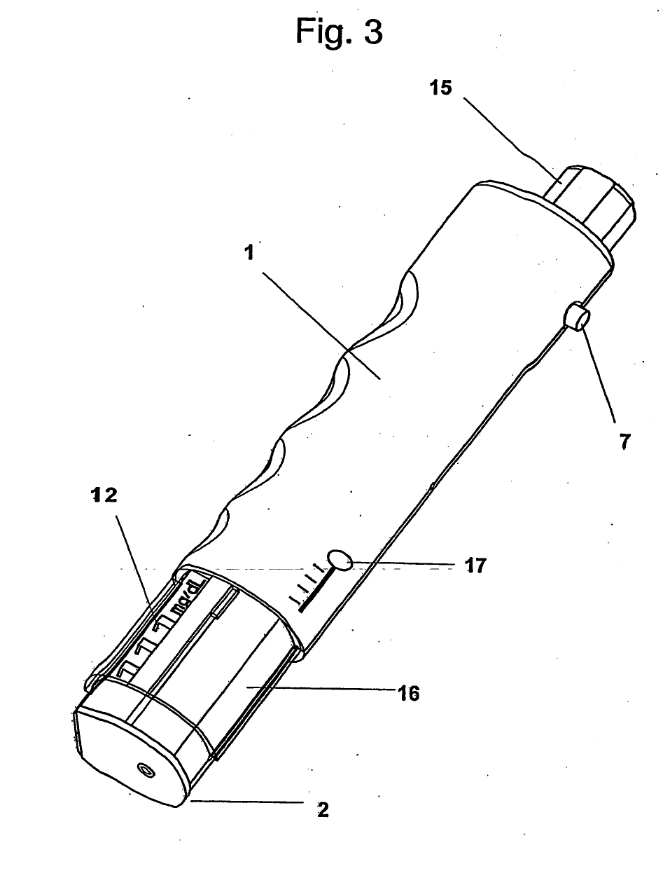

FIG. 3 is a perspective view of an apparatus according to the invention for drawing a sample and analyzing bodily fluids;

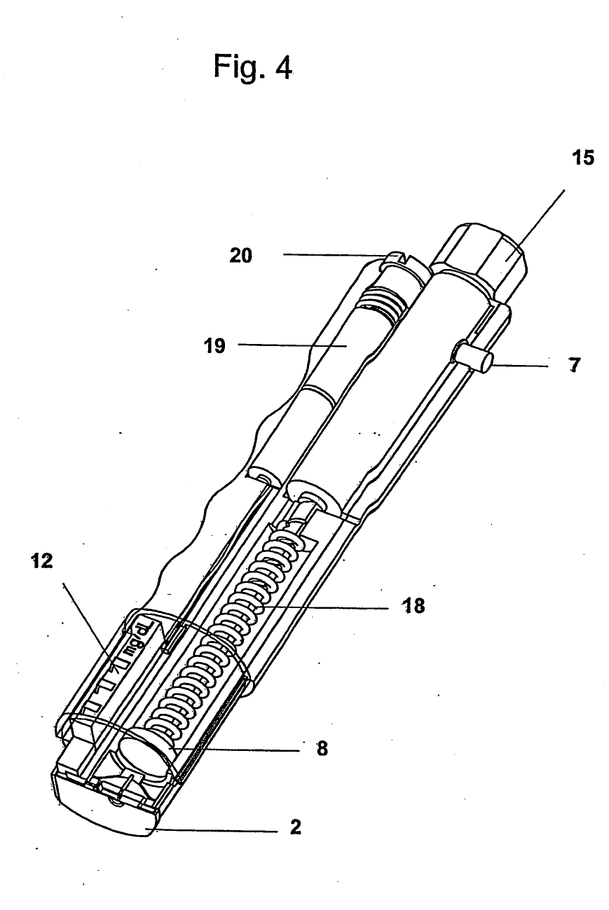

FIG. 4 is an illustration of the apparatus for drawing a sample and analyzing bodily fluids with the handle in section;

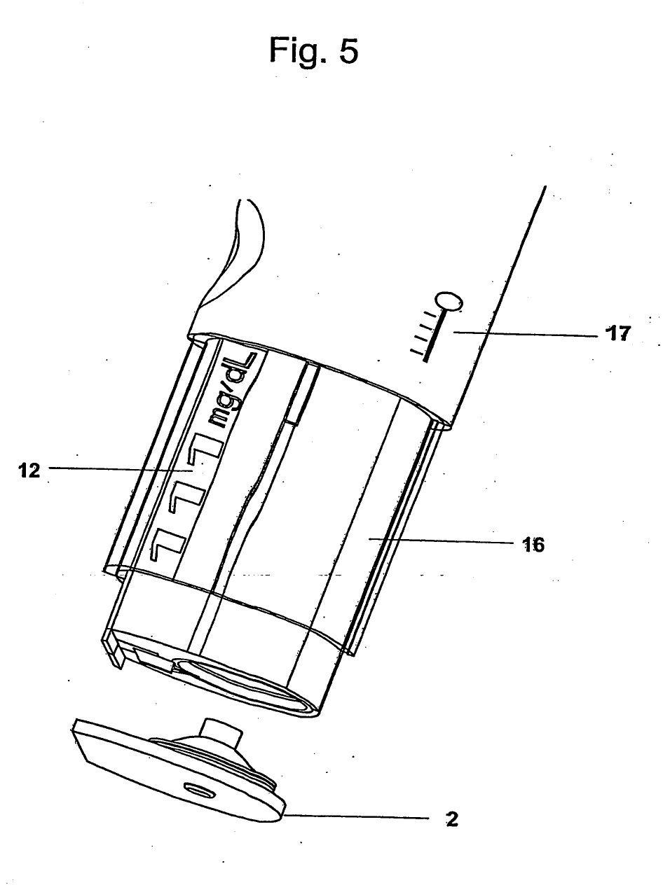

FIG. 5 shows the lower region of the handle with the sensor unit removed;

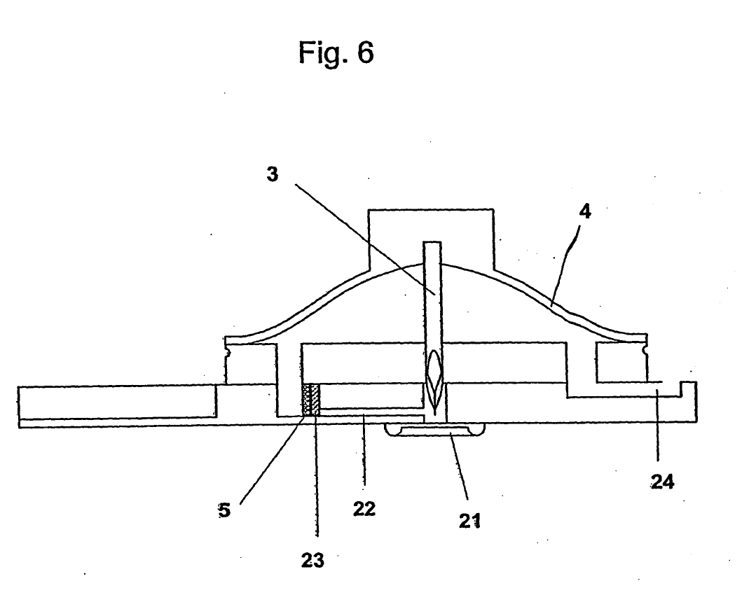

FIG. 6 is a schematic illustration of a sensor unit in longitudinal section;

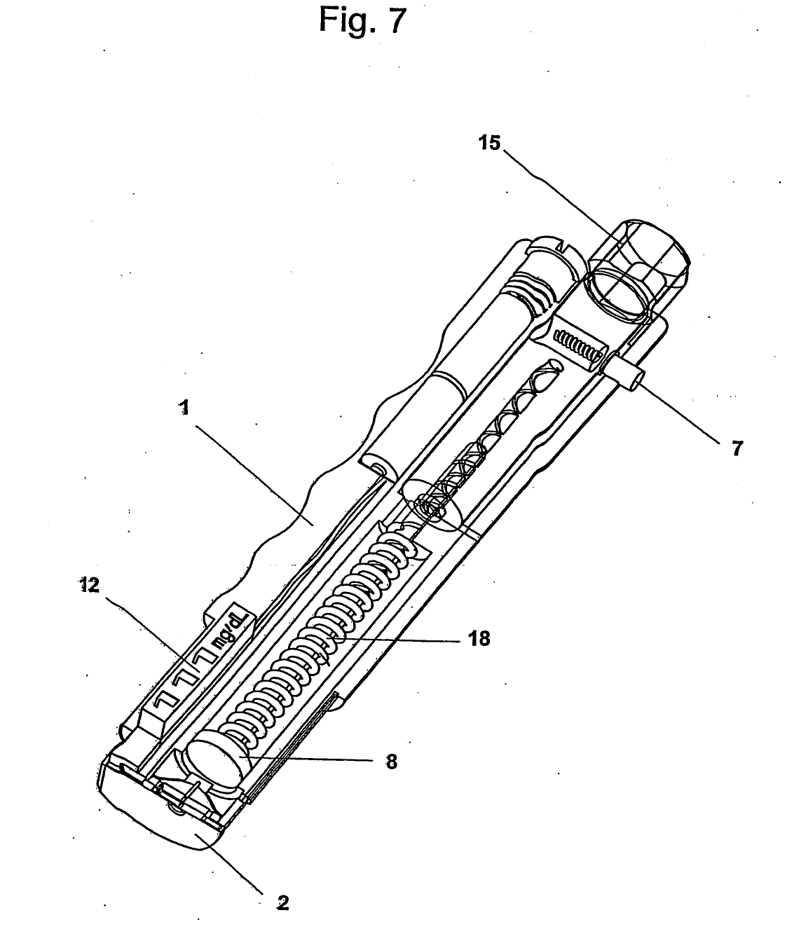

FIG. 7 is a further illustration of the apparatus with the handle in section and showing in detail the trigger mechanism;

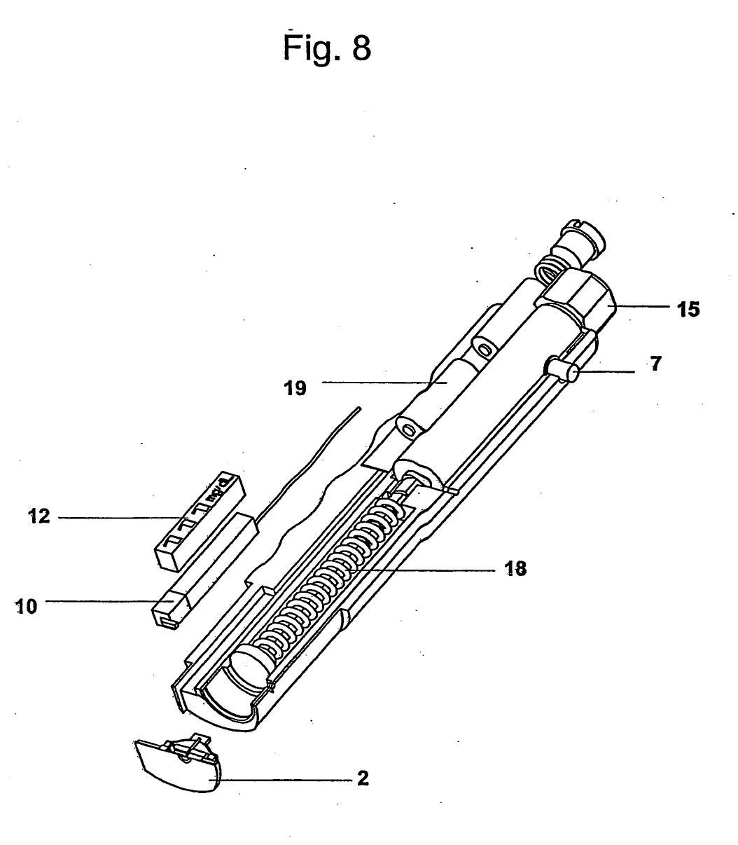

FIG. 8 shows an apparatus for drawing a sample and analyzing bodily fluids using optical measuring methods;

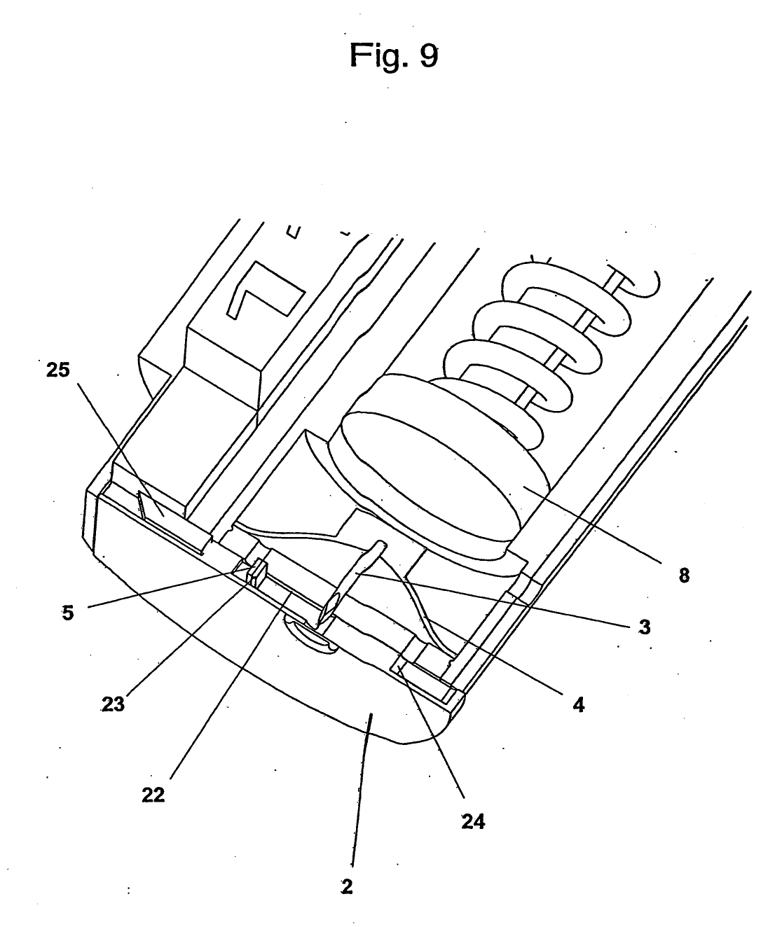

FIG. 9 is an enlarged view of the sensor unit of the apparatus according to FIG. 8;

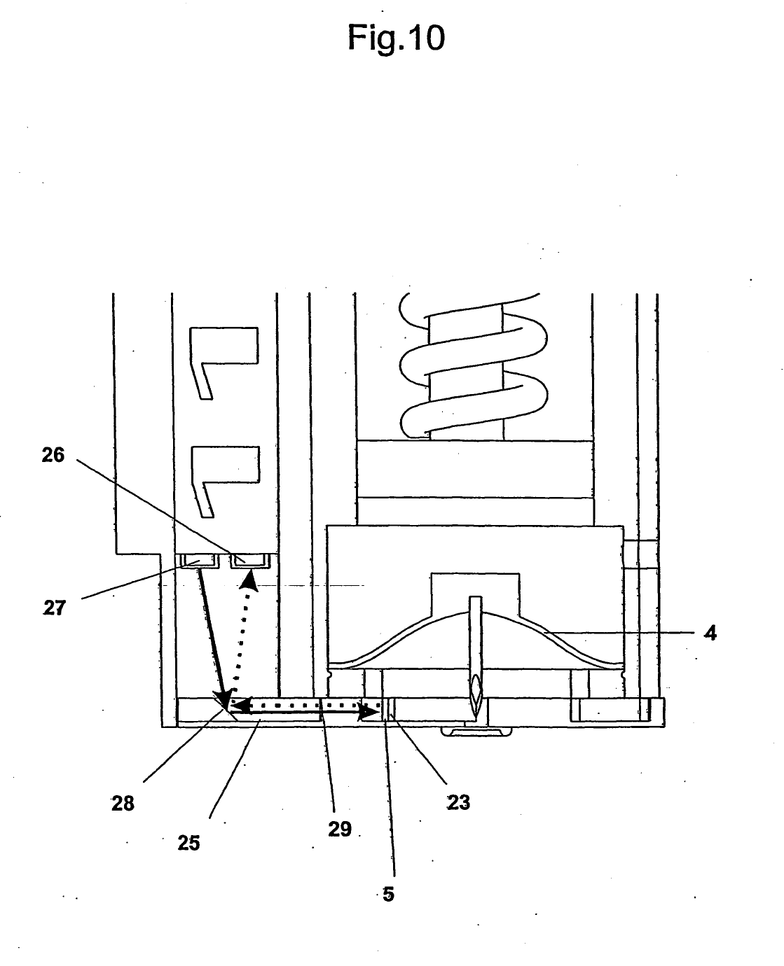

FIG. 10 is a further detailed view of the sensor unit using an optical measuring method;

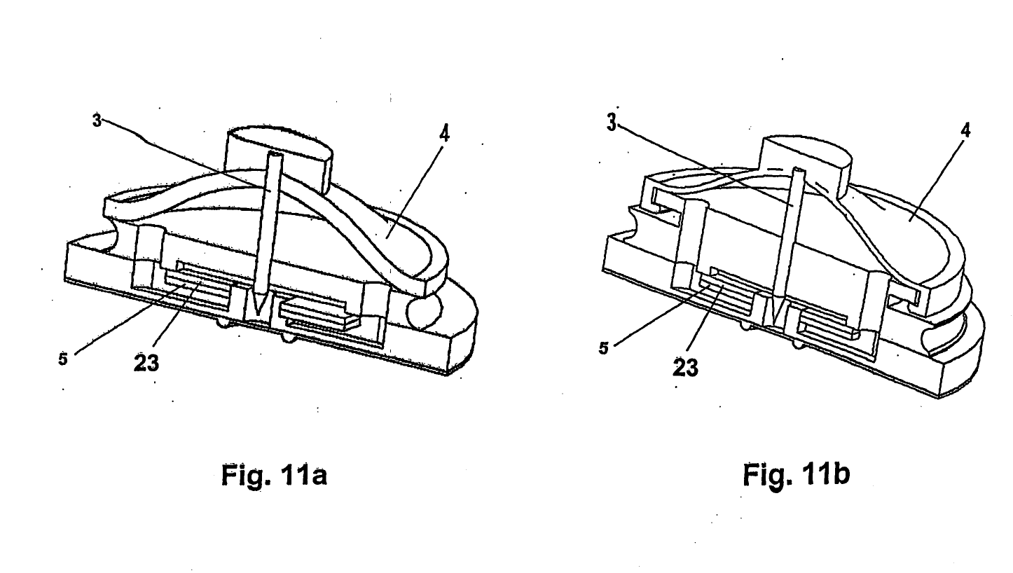

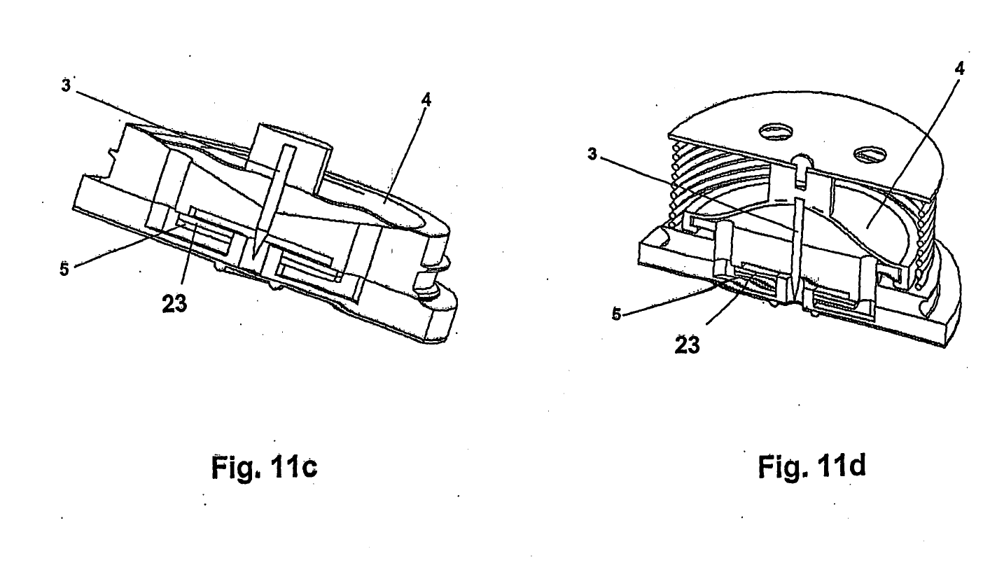

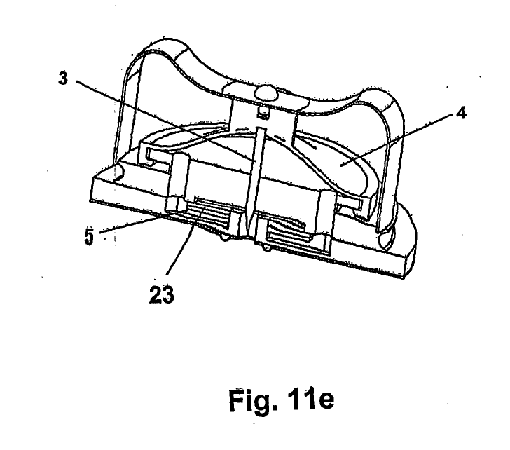

FIG. 11a to 11e are longitudinal sectional schematic illustrations of sensor units with different configurations of the elastic membrane;

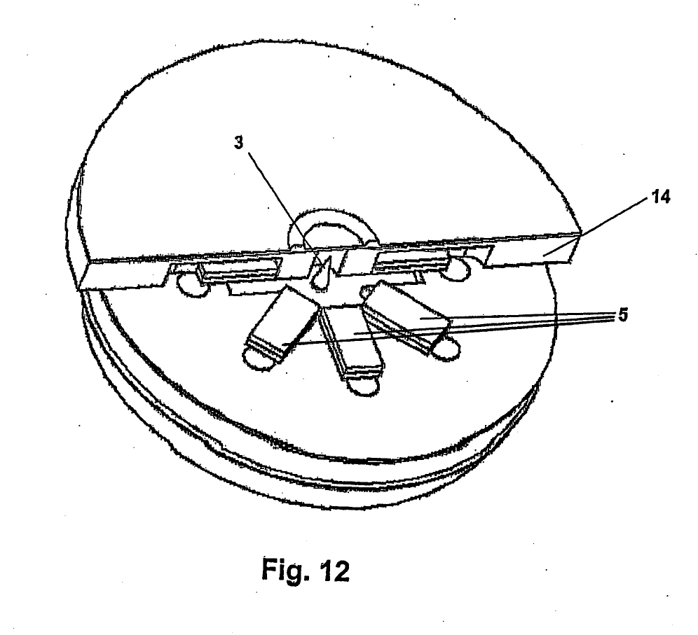

FIG. 12 is a top view onto a sensor unit with different sensor systems;

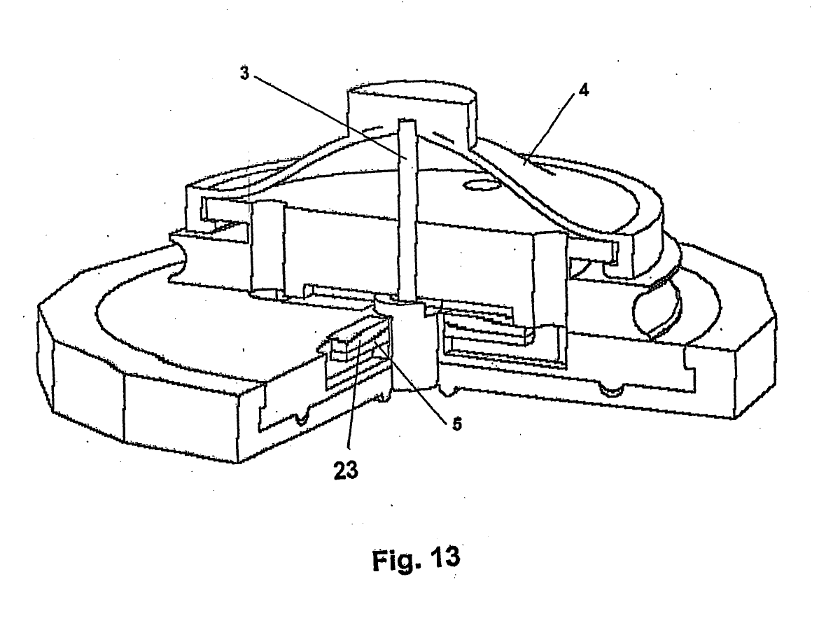

FIG. 13 is a sectional schematic illustration of a sensor unit for multiple uses;

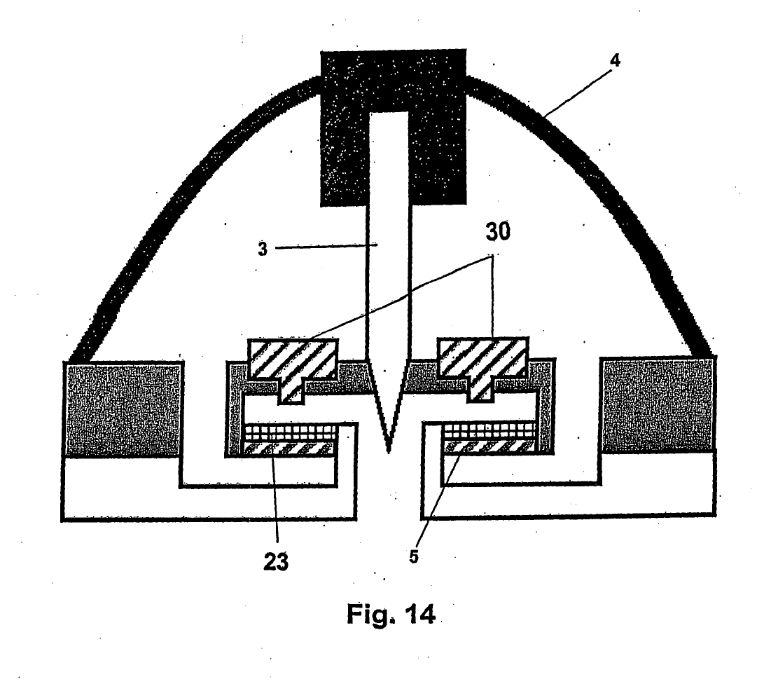

FIG. 14 is a longitudinal sectional schematic illustration of a sensor unit with an additional filter arrangement and reservoirs.

FIG. 1 shows a longitudinal sectional view of the apparatus according to the invention. The apparatus comprises a reusable handle 1 and a sensor unit 2 that is configured as a disposable item. The handle 1 holds an actuator that has a plunger 8 that is pushed downward in the housing of the handle 1 by the force of a spring 6. A lower pressure-equalizing vent opening 9 and an upper pressure-equalizing vent opening 11 ensure that the actuator can shoot forward without impairment. The actuator is actuated by a trigger 7 that the user can push in from the outside on the handle 1. Furthermore, a measuring unit 10 as well as a display 12 are provided in the handle 1. The sensor unit 2 comprises a microneedle 3 that is attached to an elastic membrane 4, allowing the microneedle 3 to be driven into the surface of the skin when pressure is exerted on the membrane 4. The sensor unit 2 and the handle 1 are connected to each other with a snap-fit connection 13. When the apparatus is used as intended, the sensor unit 2 is discarded and the handle 1 is reused. For this reason, the sensor unit 2 and the handle 1 must be separated from each other, which is done with the help of a specially provided release edge 14. The pneumatic actuator and the sensor unit 2 now mutually interact such that the downward displacement of the plunger 8 causes the elastic membrane 4 as well as the microneedle 3 attached thereto to be pushed down into the skin surface via a compressed air chamber. Excess pressure may escape afterward via the lower pressure-equalizing vent opening 9. The pressure that is exerted on the elastic membrane 4 eases, the membrane returns into its original shape, and the microneedle 3 travels upward, thus creating reduced pressure in the sensor unit 2. Consequently, bodily fluid is drawn in through the piercing location, and the bodily fluid is supplied to the sensor system 5. In the sensor system 5, the components of the bodily fluid to be determined cause a physical or chemical property change of the sensor system 5. The measuring unit 10 uses these property changes to determine concentration values for the components to be determined and shows them on the display 12.

The drawing of a sample and subsequent analysis of bodily fluid using the apparatus according to FIG. 1 will be illustrated according to eight FIGS. 2a to 2h. FIGS. 2a to 2h show different consecutive phases of the sample collection and analysis process. FIGS. 2a to 2h are identical representations of the apparatus according to FIG. 1. The reference numerals have therefore been eliminated.

FIG. 2a shows the original state of the apparatus with the reusable handle 1 and the sensor unit 2 that is configured as a disposable item and prior to use is advantageously stored in sterile packaging.

FIG. 2b shows the sensor unit 2 connected to the handle 1 by means of the latched snap-fit connection 13.

FIG. 2c shows that the spring 6 in the handle 1 is tensioned, meaning the plunger 8 is in its upper position. The trigger 7 provided on the side of the handle 1 keeps the spring 6 from releasing and pushing the plunger 8 downward.

FIG. 2d illustrates how the sensor unit 2 provided in the handle 1 is placed on the surface of the skin.

FIG. 2e illustrates the lancing phase. By actuating the trigger 7, the spring 6 relaxes and the plunger 8 moves downward. The quick movement of the plunger 8 compresses air in the chamber between the plunger 8 and the elastic membrane 4. Air can enter the handle 1 through the upper pressure-equalizing vent opening 11. The elastic membrane 4 deforms, and the microneedle 3 penetrates the surface of the skin. The air inside the sensor unit 2 is driven out through lateral slots. These slots may be configured as openings with random cross-sections and may comprise closure elements with a valve function and may open, for example, only after a predefined pressure difference has been exceeded.

FIG. 2f shows the suction phase. Compressed air escapes through the lower pressure-equalizing vent opening 9. After reaching the maximum piercing depth, the elastic membrane 4 relaxes again and returns to the original state. This creates reduced pressure inside the sensor unit 2, and bodily fluid is transported to the sensor system 5. Then, a chemical, biochemical or physical reaction takes place on the sensor system 5. As a result, a physical or chemical property change occurs in the sensor system 5.

FIG. 2g shows the actual measuring phase. The measuring unit 10 determines the physical or chemical property changes taking place in the sensor system 5, and the evaluated readings are then shown on the display 12.

After the sample collection and analysis are completed, the sensor unit 2 is removed as shown in FIG. 2h. The release edge 14 aids in separating the sensor unit 2 from the handle 1. After adding a further sensor unit 2, the handle 1 is ready for reuse.

FIG. 3 shows a perspective view of an apparatus according to the invention for drawing a sample and analyzing bodily fluids. The handle 1 has a pneumatic drive mechanism with a threaded spindle 18 and a spring that are not shown. The pneumatic actuator is tensioned by rotating a first adjustment member 15, in this example a turning knob. By pushing the trigger 7, subsequently the pneumatic actuator can be released, and air is compressed inside the handle 1. As a result, force is exerted on the sensor unit 2. The prestressing of the spring on the threaded spindle 18 in the pneumatic actuator tensions can be varied by rotation of the knob 15. This way, the piercing depth can be variably adjusted. A second adjustment member 17 may be used to adjust the piercing speed. Furthermore, a measuring unit 10 that is not visible here, is integrated in the handle, which unit can determine the physical or chemical property changes in the sensor system 5. The determined values are then displayed on the display 12. Electrical energy can be obtained when using an appropriate piezoelement while tensioning the pneumatic actuator. Electric power, however, may also be produced electromagnetically by means of a small magnet that has the advantage that then the relaxation phase of the spring on the threaded spindle 18 may be utilized. It is preferred if the electrical energy is obtained with the second method.

The obtained electrical energy may then be used, for example, for a control unit for detecting the correct filling level with bodily fluid or for supplying power to self-sufficient sensors. FIG. 3 furthermore shows a sleeve 16 for releasing the sensor unit 2 after use.

FIG. 4 offers a view of the inside of the apparatus for drawing a sample and analyzing bodily fluids. The measuring unit 10 is integrated in the handle 1, and the sensor unit 2 has been installed. Upon actuating the trigger 7, the threaded spindle 18 with the spring located on the inside of the handle 1 drives the plunger 8 downward, and at the same time compresses the air column located above the elastic membrane 4 in the sensor unit 2 and thus drives the microneedle 3 integrated in the sensor unit 2 downward. In this example, power is supplied by batteries 19 in a specially provided battery compartment 20.

FIG. 5 shows the lower portion of the handle 1 with the sensor unit 2 removed. The unit had been attached to the handle 1 via a snap-fit connection 13 that is not shown here, and was subsequently separated from the handle 1 by means of pressure via the sleeve 16 on the release edge 14. The sensor unit 2 is typically intended for single use.

FIG. 6 is a longitudinal sectional schematic view of the sensor unit 2. When applying external pressure on the elastic membrane 4, the microneedle 3 attached in the elastic membrane 4 moves downward and penetrates the surface of the skin, provided that the handle 1 with the sensor unit 2 was previously placed on the skin surface. Air escapes during this step, to which end for example a ventilation passage 24 may be configured as a check valve that allows air to flow only in one direction, namely from the inside to the outside. After the external pressure on the elastic membrane 4 is reduced, the microneedle 3 retracts, creating reduced pressure in the sensor unit 2 and drawing bodily fluid into the sensor unit 2. The fluid is guided directly onto a filter arrangement 23 through a capillary 22, is filtered and then comes in contact with the sensor system 5. A sealing ring 21 serves to seal the sensor system 5 in relation to the skin surface. In a special embodiment, the sealing ring 21 may be provided with an adhesive layer to ensure better contact with the skin. The bottom of the sensor unit 2 may comprise a removable protective film, which has already been removed in this example, to keep the unit sterile until use. In another embodiment, the protective film may remain on the bottom of the sensor unit 2 during piercing, however it must be pretensioned to ensure that a sufficiently large opening is created during piercing.

FIG. 7 illustrates a further apparatus with the handle 1 in section and the trigger mechanism shown in detail. By pressing the trigger 7, the threaded spindle 18 with spring is released and drives the plunger 8 downward.

FIG. 8 shows an apparatus for drawing a sample and analyzing bodily fluids, which apparatus uses optical measuring methods. Particularly fluorescence spectrometry methods are used. The measuring unit 10 includes a light source 27 and a photodiode 26 as well as the necessary electronics. A battery 19 provides the power. On the outside of the handle 1, a display 12 is provided for displaying the obtained readings.

FIG. 9 is an enlarged view of the sensor unit 2 of the apparatus according to FIG. 8. A bundle of optical fibers 25 is provided on the bottom of the handle 1 and firmly connected thereto. It conducts the excitation light originating from the light source 27 directly to the sensor system 5, collects the emitted light again and then directs it to a photodiode 26 in the measuring unit 10, which photodiode is not shown here.

FIG. 10 shows a further detailed view of the sensor unit 2 when using an optical measuring method. The excitation light exits the light source 27 and is directed via a lens 29 onto the sensor system 5 by being fully reflected on a surface 28. The light emitted by the sensor system 5 is directed to the photodiode 26 via the lens 29 and the fully reflective surface 28. The entire optical arrangement is preferably configured as an integrated optical component.

FIGS. 11a to 11e show longitudinal sectional schematic illustrations of a sensor unit 2 with different configurations of the elastic membrane 4. In all cases, the microneedle 3 attached to the elastic membrane 4 penetrates through the surface of the skin when external pressure is exerted on the elastic membrane 4. FIG. 11b shows one embodiment of a sensor unit 2 in which the elastic membrane 4 is fitted to an injection-molded part. When external pressure is exerted on the elastic membrane 4, air escapes laterally from the sensor unit 2 through the latch. When the elastic membrane 4 relaxes, reduced pressure builds in the sensor unit 2 and as a result the bodily fluid is drawn out of the skin. A further embodiment is shown in FIG. 11c, in which a planar elastic membrane 4 and a corresponding recess are used, allowing the volume to be varied analogously to the elastic membranes 4 of the other sensor units 2. The elastic membrane 4 may be held in its shape alternatively by a spring or a bracket, as is shown in FIGS. 11d and 11e.

FIG. 12 shows a top view onto a sensor unit 2 with different sensor systems 5. This way, detection systems for different test samples are provided, the test samples being detected either simultaneously or in a defined sequence over time.

FIG. 13 is a sectional schematic illustration of a sensor unit 2 for multiple use. By using a rotatably latching feed passage for the bodily fluid, particularly blood, the flow is only directed to one sensor system 5. After the test samples have been measured, the feed passage rotates further by one notch and opens the blood passage to the next sensor system 5. This way, one sensor unit 2 can be used for multiple determinations of the same test sample.

FIG. 14 shows a longitudinal sectional schematic illustration of a sensor unit 2, where the quantitative determination of test samples in the bodily fluid occurring after drawing the bodily fluid is carried out only after the sample has been appropriately processed. The filter arrangement 23 may hold substances for preparing the samples. Furthermore, reservoirs 30 with substances for preparing the bodily fluid sample and/or for rinsing may be provided, these substances being released, for example by applying pressure via valves and membranes. In both cases, correct coverage of the sensor matrix may be ensured by measuring resistivity.

Compared to existing methods, the drawing of samples and analyzing of bodily fluids carried out with the apparatus according to the invention and advantageous embodiments thereof and with the method offer the following advantages: The volumes of bodily fluid required are low and largely pain-free lancing is guaranteed by using microneedles. Manufacturing costs are low as a result of making the sensor unit in an injection-molding process. Use is comfortable since the lancing unit and the sensor system are integrated into one unit for single use and the measuring unit is integrated in the apparatus. Furthermore, the integration principle ensures maximum hygiene. The apparatus has an ergonomic shape and low weight due to minimized power consumption.

1. An apparatus for drawing a sample and analyzing bodily fluids, comprising
a handle holding a pneumatic actuator, and
a sensor unit that comprises a microneedle attached to an elastic membrane and at least one sensor system, the handle and the sensor unit being detachably connected to each other and the pneumatic actuator exerting pressure on the elastic membrane so that the microneedle pierces the skin and subsequently, as the pressure is reduced, the elastic membrane returns to its original shape and thus produces reduced pressure in the interior of the sensor unit, as a result of which bodily fluid is directed to at least one sensor system and causes a physical or chemical property change of the sensor system.

2. The apparatus according to claim 1, characterized in that the handle comprises a measuring unit for determining the physical or chemical property changes caused in the sensor system.

3. The apparatus according to claim 2, characterized in that the measuring unit uses the physical or chemical property changes to determine at least one concentration value.

4. The apparatus according to claim 3, characterized in that a display provided on the apparatus shows the concentration value determined by the measuring unit.

5. The apparatus according to claim 2, characterized in that at least one sensor system and the measuring unit are designed for an optical measuring method.

6. The apparatus according to claim 5, characterized in that at least one sensor system and the measuring unit are designed for a fluorescence spectroscopic measuring method.

7. The apparatus according to claim 6, characterized in that at least one sensor system and the measuring unit are designed for a fluorescence spectroscopic measuring method on the basis of fluorescence resonance energy transfer.

8. The apparatus according to claim 1, characterized in that the sensor unit comprises several sensor systems.

9. The apparatus according to claim 8, characterized in that the sensor unit has a rotatably latching feed passage for the bodily fluid, so that the bodily fluid is only directed to one sensor system and upon completed measurement of a test sample the feed passage is rotated further by one notch to open the access for bodily fluid to the next sensor system.

10. The apparatus according to claim 1, characterized in that a detachable connection between the handle and the sensor unit is configured as a plug, clamping, snap-fit, screw or adhesive connection.

11. The apparatus according to claim 1, characterized in that the sensor unit comprises a control unit for detecting the correct, particularly sufficient, filling level with bodily fluid.

12. The apparatus according to claim 11, characterized in that the control unit comprises an electric resistivity measuring unit.

13. The apparatus according to claim 11, characterized in that the control unit defines a filling volume of less than 0.5 microliters as the target value for a sufficient filling level.

14. The apparatus according to claim 1, characterized in that the electric energy required for the components of the apparatus is produced by coupling piezoelectric or electromagnetic elements to the pneumatic actuator.

15. The apparatus according to claim 1, characterized in that the sensor unit comprises a sealing ring on the bottom.

16. The apparatus according to claim 15, characterized in that the sealing ring has an adhesive layer on the bottom.

17. The apparatus according to claim 1, characterized in that the sensor unit is closed on the bottom in a sterile fashion by a removable or a pretensioned tearable cover film.

18. The apparatus according to claim 1, characterized in that the handle has a first adjustment member for variably defining the piercing depth of the microneedle.

19. The apparatus according to claim 18, characterized in that the handle has a second adjustment member for variably defining the piercing speed of the microneedle.

20. The apparatus according to claim 19, characterized in that the handle has a third adjustment member for variably defining the suction speed of bodily fluid in the sensor unit.

21. The apparatus according to claim 1, characterized in that a filter arrangement is provided upstream of at least one sensor system.

22. The apparatus according to claim 1, characterized in that one or more reservoirs with reagents and/or rinsing solutions are provided upstream of at least one sensor system.

23. A method for drawing a sample and analyzing bodily fluids, comprising the following steps:
a pneumatic actuator accommodated in a handle exerts pressure on an elastic membrane in a sensor unit so that a microneedle attached to the elastic membrane pierces the skin,
after reducing the pressure, the elastic membrane returns to its original shape and thus produces reduced pressure in the interior of the sensor unit, as a result of which bodily fluid is directed to at least one sensor system and causes a physical or chemical property change there of the sensor system.

24. The method according to claim 23, characterized in that in a further step the physical or chemical property changes caused in at least one sensor system are used by a measuring unit provided in the handle to determine a concentration value.

25. The method according to claim 24, characterized in that in a further step the concentration values determined by the measuring unit are displayed on a display.

26. The method according to claim 24, characterized in that in a further step the concentration values determined by the measuring unit are transmitted to an external apparatus by means of a transponder or transmitter.

27. The method according to claim 26, characterized in that the external apparatus is an automatic syringe or a pump for administering appropriate therapeutic agents.

28. The method according to claim 26, characterized in that the external apparatus is a display unit, a mobile telephone, a watch, a computer or a PDA.

29. The method according to claim 23, characterized in that the apparatus according to claim 4 is used.
